(19) United States
(12) Patent Application Publication
(10) Pub. No.: US 2023/0241298 A1
(43) Pub. Date: Aug. 3, 2023

(54) Breast Pump Insert

(71) Applicant: Crystal Nelson, Redmond, OR (US)

(72) Inventor: Crystal Nelson, Redmond, OR (US)

(21) Appl. No.: 18/105,489

(22) Filed: Feb. 3, 2023

Related U.S. Application Data
(60) Provisional application No. 63/306,374, filed on Feb. 3, 2022.

(51) Int. Cl. A61M 1/06

(52) U.S. Cl. CPC A61M 1/066

(57) ABSTRACT

A breast flange adapter is provided to adapt conventional flanges provided with conventional breast pumps to the breast of the user. The flange adapter can be provided in a kit having a plurality of such flange adapters of differing sizes. The flange adapters are configured at a first or engagement end for operative sealed engagement with the provided breast flange of conventional breast pumps.

### FIELD OF THE INVENTION

This application claims priority to U.S. Provisional patent application Ser. No. 63,306,374 filed on Feb. 3, 2022, which is incorporated herein in its entirety by this reference thereto.

The present application relates to breast feeding of infants by their mothers or caretakers. More particularly, it relates to an insert which is configured to operatively engage to the existing inner flange of a conventional portable breast pump, to configure it to a determined size of the breast of a woman employing the breast pump.

### BACKGROUND OF THE INVENTION

Breast feeding, as is conventionally well known, is the natural process of feeding breast milk by the mother to her infant. While this process can be accomplished in real time directly from mother to infant, frequently, especially with working mothers, it is accomplished by expressing milk from the breast of the mother and storing the milk for subsequent use where it may be employed in bottle-feeding of the expressed breast milk to the infant.

Such milk expression is conventionally accomplished by a breast pump device. In many cases, such breast pumps are portable, such as those manufactured and sold by the Willow Company of Mountain View Calif., and the ELVIE portable breast pump by Chiaro Technology Limited in the United Kingdom.

Conventionally, such milk gathering and storing sessions for expressing breast milk employ a breast pump which is configured with a flexible flange. This flange is intended to sealably engage with the breast of the mother during use. A mechanical or other means to pump is operatively engaged to the flange which is placed in contact with one or both breasts of the user to thereby express milk. The expressed milk is routed, by the pumping device, to deposit within a container for storage and later use for feeding sessions.

When employing a breast pump to express milk, it is an important consideration, for both user comfort and maximizing the volume of expressed milk, to employ a flexible flange in proper contact with the breast. A conventional breast pump flange, as provided with most conventional stationary and portable (user worn) breast pumps, should fit comfortably around the nipple of the breast of the user in a substantially sealed contact prior to and during pumping by the engaged breast pump.

However, most manufacturers of such breast pumps only provide a single such flexible flange, which is intended to operatively and comfortably engage with the breast of the user. Such a comfortable fit is frequently not the case and users can be subjected to significant discomfort as well as poor expression of milk from ill-fitting flanges which are not properly sized for comfortable and productive operative contact with the breast.

A comfortable and properly fitting flexible breast pump flange should insure that during use there is no discomfort and that an operative seal is continuously accomplished with and to the breast of the user in order to provide for optimum suction to the breast and maximized milk expression. Further, such a comfortable and proper fit will also help to ensure that when the mother later breast feeds the infant directly, that there is no tenderness or other discomfort.

The forgoing description concerning breast pumps employ for milk expression is intended to be illustrative and not exclusive. As such, no limitations on the invention described and claimed herein is to be associated with such. Further, additional limitations and features of the current art of breast pumps are well known, or such will become apparent to those skilled in the art upon a reading and understanding of the specification below and the accompanying drawings.

### SUMMARY OF THE INVENTION

The breast pump flange system and device herein, in various modes, provides for the adaptation of existing flexible flanges of breast pumps, such as the ELVIE and the WILLOW and similar worn breast pumps, to a fit of a proper size for the actual breast size of the user. For example, conventionally, a widely employed “standard” size for the opening of a flexible flange is formed to accommodate a breast nipple of a diameter which is substantially 24 mm. However, many women have nipple diameters which are smaller than the conventionally provided flexible flange.

The device herein is a solution to such poor fitting breast pump flanges through the provision of one or a plurality of flange adapters. Each such flange adapter is formed of flexible polymeric material such as polyethylene or silicone to thereby provide a soft comfortable operative contact against the breast of the user. Such soft polymeric material also insures that a good seal is maintained during the expression session.

A first end or engagement end of each flange adapter is configured with an exterior circumferential surface which is adapted is size to form a sealed engagement with the surface of the internal circumference of the passage of the existing breast pump flange. This sealed configuration ensures that any suction communicated to the engaged end of the existing breast pump flange will be fully communicated to the internal passage of the flange adapter herein.

A second end of the flange adapter device herein is formed with a larger diameter than the that of the first end. It is formed in a frusto-conical shape where the wider end thereof is positionable to form a seal on the breast of the user in the area of the breast which surrounds the nipple.

An axial passage communicates through the flange adapter from the first end where the axial passage is in a sealed communication with the suction communicated to the flexible flange provided with and mounted to the conventional breast pump. It has been found during experimentation that forming the diameter of this axial passage, in each of the flexible adapter flanges provided herein, in a size to comfortably engage around and with the nipple of the user has both comfort and operational benefits. As such, providing the kit with varying sized axial passages is particularly preferred to allow each user to choose a flange adapter for the day of use, which is most comfortable. It was found that due to daily changes in breast sizes of users, that the chosen adapter flange may vary, and the provision of the flange adapters in the kit form is preferred to accommodate daily choice.

Thus, when the device herein is provided in a preferred mode as a kit of available flange adapters, each will provide the multiple flange adapters having the same exterior diameter at the end where the circumferential surface thereof is sized to sealably engage with the existing internal passage of the flange of the conventional breast pump. However, each respective axial passages of each of the multiple or plurality of available adapter flanges is preferably formed with a different diameter of the internal axial passage.

Currently, such internal axial passages are provided in diameters from 13 mm to 19 mm in increments increasing by 2 mm from the smallest to the largest diameter. However, more sizes can be provided in increasing increments of 1 mm from 13 mm to 19 mm or more. To render it easy for the user to pick the properly sized flexible flange adapter herein, indicia relating to the actual size of the internal axial passage is preferably embossed into the flexible polymeric material forming the flange adapter in a font size easily read without reading glasses or in dim light. This makes it easy for the user to employ existing light or without the need for reading glasses and to discern the size of the axial passage of each respective flange adapter. The kits are preferably provided to users in a single unit. However, it is anticipated that individual flange adapters of differing sizes can be provided to augment those in the initial kit to allow for customization.

During experimentation to construct a flange adapter for engagement to conventional breast pumps, it was found that the communication of suction to conventional breast pumps varied in position. Initially, this caused issues where the prototypes are engaged with some conventional pump devices. Further, it was found that in some cases, users would change the brand of conventional breast pump which on occasion rendered the flange device herein inoperative.

After a number of configuration adaptations it was found that an optional removable section could be provided at the first end of some or all of the members of the kit of the flange adapters. The provision of this removable section was found to accommodate brands of portable breast pumps where the suction is communicated to the internal sidewall. While such a side-positioned configuration will work where there is no flange adapter inserted, in such a configured breast pump, an opening through the sidewall of the flange adapter to the axial passage is required to communicate the suction to the breast of the user when operatively engaged with the flange adapter herein.

To that end, in the plurality of flange adapters herein, it was found that the communication of suction to the axial passage of the flange adapter could be provided in the form of one or a plurality of removable portions of the sidewall of the flange adapter herein. This provision of one or more removable sections was found to allow the chosen flange adapter to be operatively engaged with breast pumps which communicate suction to an opening in the existing flange which aligns with the axial passage of the flange adapter herein at the first end thereof. Should, however, it be determined that the suction passage does not align and is communicated through the sidewall of the existing flange, or off center from the axial passage thereof, then the user has the option of removing the portion of the sidewall to form the opening therein. The removable portion can be rendered frangible by forming a recess which is cut in the shape of the removable portion, which does not completely sever the sidewall. The sidewall, thus, remains sealed for use with breast pumps having suction from a passage aligned with the axial passage of the flange adapter. However, for those conventional breast pumps having an off-center communication of suction, or suction from a side surface, the removable portion allows for use with such by cutting or tearing the remaining material or tearing it.

An additional preferred mode of the flange device herein will also provide removable end portions. It was found during experimentation in efforts to form a flange device engageable with all conventional breast pumps, that different brands of breast pumps can have length differences with regard to the internal passage of the existing breast flange. Some, it was found, require a longer first end on the flange adapter device herein to operatively seal with the existing internal passage. It was also found, with some brands, that a shorter first end of the flange device is required for a good seal and positioning.

As such, in addition to the removable sections, noted above to accommodate some breast pump configurations, it was found by rendering portions of the first end of the flange adapter herein easily removable, in the same fashion as the removable openings, the user will be able to adapt the flange adapter device herein to a length to operatively engage with the internal passage of substantially any existing flange of conventionally sold breast pumps.

With regard to the above disclosure, those skilled in the art will appreciate that the conception upon which this disclosure is based, once this specification is read, may readily be utilized as a basis for designing other breast flange sizing systems and for carrying out the several purposes of the present disclosed device using equivalent structures and components. It is important, therefore, that the claims herein be regarded as including such equivalent construction and methodology which those skilled may employ subsequent to a review of this specification, insofar as they do not depart from the spirit and scope of the present invention.

As used in the claims to describe the various inventive aspects and embodiments, “comprising” means including, but not limited to, whatever follows the word “comprising”. Thus, use of the term “comprising” indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements. The term “substantially” when employed herein, means plus or minus twenty percent unless otherwise designated in range.

It is an object of the present invention to provide flange adapters which will provide the user a means for alteration of the diameter of the existing internal passage of breast pump flanges to one matching the diameter of the nipple of the user.

It is a further object of the invention herein to provide a flange adapter for the flange of conventional breast pumps, which is configured at a first end for operative engagement with breast pump flanges varying in size, length, and the position of suction communication.

These and other objects, features, and advantages of the presently disclosed breast pump flange adapters system herein as well as the advantages thereof over existing prior art, which will become apparent from the description to follow, are accomplished by the improvements described in this specification and hereinafter described in the following detailed description which fully discloses the invention, but should not be considered as placing limitations thereon.

### BRIEF DESCRIPTION OF DRAWING FIGURES

The accompanying drawings, which are incorporated herein and form a part of the specification, illustrate some, but not the only or exclusive examples of embodiments and/or features of the disclosed breast pump flange adapter. It is intended that the embodiments and figures disclosed herein are to be considered illustrative of the invention herein, rather than limiting.

In the drawings:

FIG. 1 shows an existing portable breast pump and the conventional flexible flange provided therewith for engagement to the breast of the user and further shows an example of the flange adapter device herein positioned for insertion into the internal passage of such an existing flange.

FIG. 2 shows the flange adapter herein having an exterior diameter which sizes the circumference at the first end of the flange adapter device and also shows annular sealing ridges having a formed channel therebetween, adapting the device for forming a seal with the interior circumference of the wall defining the internal passage of an existing flange.

FIG. 3 depicts a flange adapter herein having an axial passage sized to comfortably and operatively engage with the nipple of a user having a 15 mm diameter and having the exterior circumference similar in size to that of FIG. 1.

FIGS. 4-5 show other configurations of the flange adapter herein wherein the exterior circumference is similar to that of the device in FIG. 1, but the diameter of the axial passage of the flange adapters varies between 17 mm to 19 mm.

FIG. 6 depicts the flange adapter herein showing a removable section in the sidewall thereof positioned at the first end thereof within the channel between the two ridges.

FIG. 7 shows the flange adapter herein in a mode with removable sections thereof positioned at the first end thereof to thereby provide for adjusting the length and showing removable sections in each removable portion which can be separated if necessary to accommodate side-positioned or off center suction in the internal passage of the existing flange.

FIG. 8 shows the first end of the flange adapter herein and examples of preferred dimensions thereof and also shows the opening formed in the sidewall which would be positioned to communicate suction to the axial passage of th flange adapter herein.

FIG. 9 shows a chart of sidewall thicknesses which yield varying diameters of the axial passage of the flange adapter herein, while maintaining the external circumference of the first end, to thereby sealably engage within the diameter of the internal passage of the existing flange on the breast pump.

### DETAILED DESCRIPTION OF THE INVENTION

In this description, the directional prepositions of up, upwardly, down, downwardly, front, back, top, upper, bottom, lower, left, right, front, back, and other such terms refer to the device as it is oriented and appears in the drawings and are used for convenience only and such are not intended to be limiting in any fashion, or to imply that the device has to be used or positioned in any particular orientation.

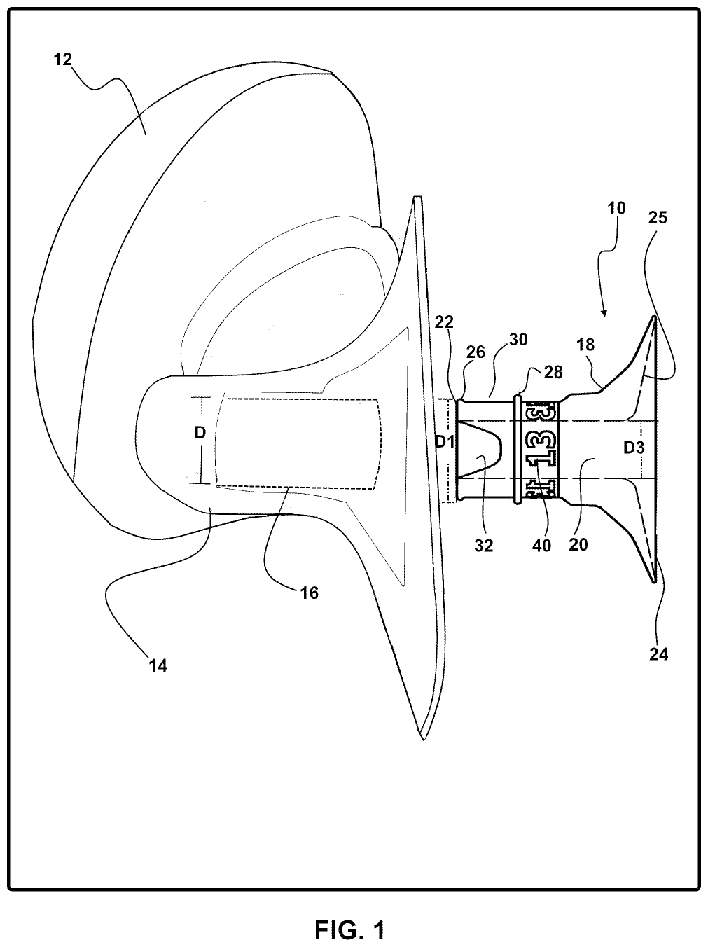

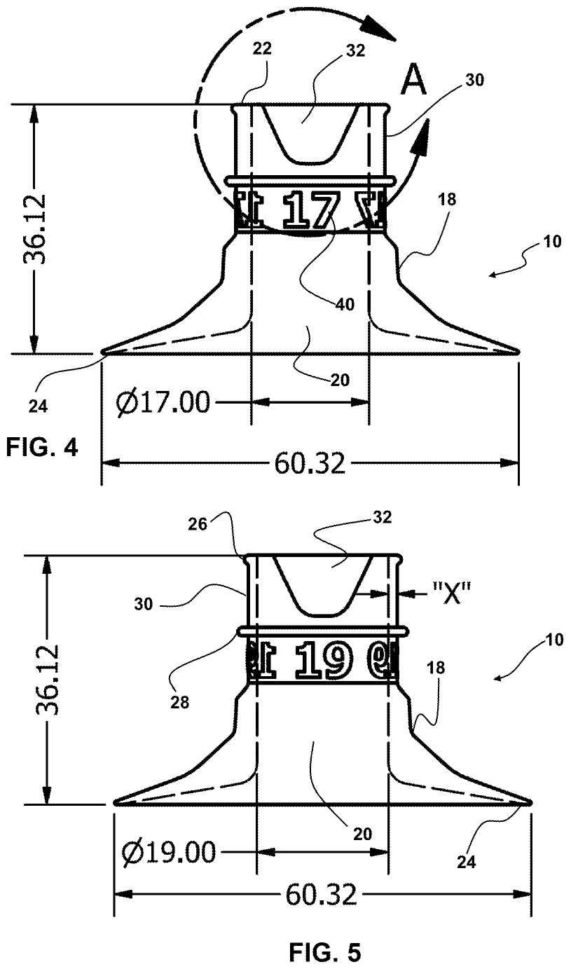

Now referring to drawings in FIGS. 1-9 wherein similar components are identified by like reference numerals, there is seen in FIG. 1 an example of a conventional existing portable breast pump 12 which is sold for both portable and non portable use, where it is enclosed in a nursing bra and engaged to the breast of the user, as well as modes of such pumps 12 which are counter-positioned or otherwise used at home.

Such breast pumps 12 conventionally have a flexible provided flange 14 which is provided to the user with a pump 12 which is operatively engaged with that provided flange 14 and which is intended for direct operative engagement with the breast of the nursing mother. As noted, such conventional flexible provided flanges 14 are provided with a fixed diameter internal passage 16 which is intended to be a one size fits all configuration where the diameter “D” of the provided internal passage 16 is intended to operatively engage around the circumference and with the nipple of the breast of the nursing mother.

As noted, this single sized diameter “D” of the conventional flexible provided flange 14, while easing the requirements of the manufacturer, does not always serve the user well. This is because the diameter and thus the circumference of the nipple of the breast of nursing mothers, can vary widely. Thus, the provision of the flexible flange 14, with a single diameter “D”, yielding a single size to the internal circumference of the internal passage 16 may not provide a comfortable and operative fit to many users who may have smaller diameter nipple sizes than that of the diameter “D” of the internal passage 16 of the provided flexible flange 14.

The flange adapting device 10 and system herein, when employed in combination with the provided flange 14 of a conventional portable or in home breast pump 12, allows the user to adjust the diameter “D” of the internal passage 14 of the conventional flexible provided flange 14 to one which is more comfortable.

The user, such as a nursing mother, will choose and employ a flange adapter device 10 formed with a body 18 which is substantially cylindrical which has a diameter D1 yielding an exterior circumference thereof at the first end 22, which is configured to removably sealably engage with the internal passage 16 of the provided flexible flange 14. This removably sealed engagement allows the user to remove the flange adapter device 10, as needed, for both cleaning as well as the ability to choose a different flange adapter device 10 with a different sized axial passage, should their needs change as to the size of their nipple on any given day.

In use, the device 10 may be sold in varying sizes for forming members of a kit of flange devices 20 or as a kit including multiple flange adapter devices 10. The user will choose a flange adapter device 10 from those available having a body 18 which has the diameter D1 which will form an external circumference configured for a sealed removable engagement within the internal passage 16 of the provided flexible flange 14 of the breast pump 12. The provision of adapter devices 10 having a first sealing ridge 26 and a second sealing ridge 28 forming a recess or an annular channel 30 therebetween, was found in experimentation to form a better sealed engagement of the first end 26 with provided flexible flanges 14 from a wider number of differing manufacturers.

Once a flange adapter device 10 having a body 18 with an axial passage 20 of a diameter D3, determined to be both a comfortable and operable fit to the nipple of the user, has been chosen from the plurality thereof available, the first end 22 thereof will be slid into removable sealed engagement with the internal passage 16 of the conventional flexible provided flange 14. Thereafter, the user will slide the axial passage 20 sized for the diameter and circumference of the nipple of their breast, to a removable engagement therearound in an as-used positioning. With the body 18 of the chosen flange adapter device 10 in this as-used positioning, the suction communicated to the internal passage 16 of the flexible provided flange 14 is communicated to the axial passage 20, whereupon milk is expressed and drawn into a reservoir of the breast pump 12. A frusto-conical sealing flange 25 surrounding the second end of the axial passage 20 is preferred to provide a larger contact area with the skin of the breast surrounding the nipple and thus a better seal with the breast of the user.

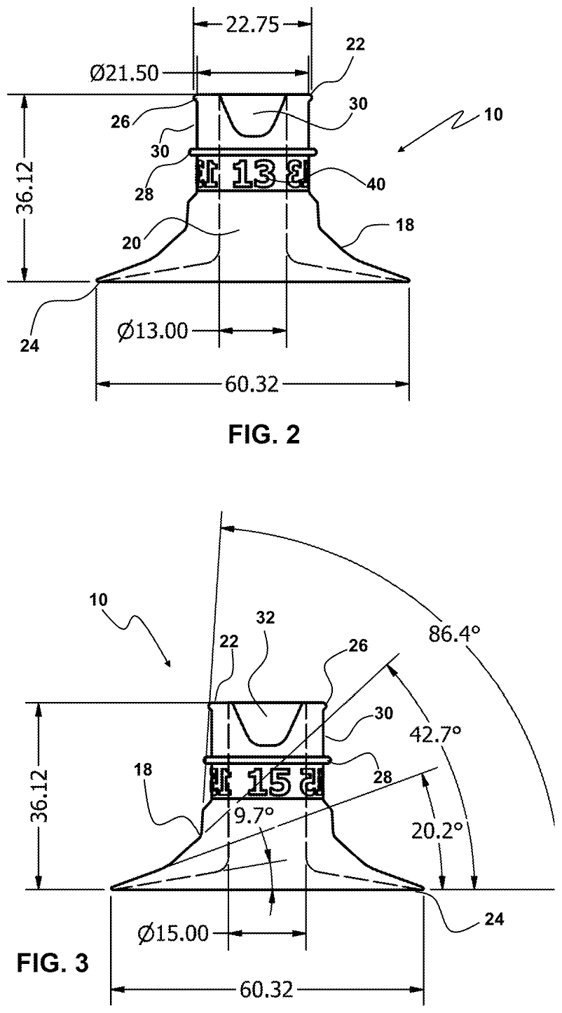

As shown in FIGS. 2-5, the flange device 10 herein is provided such that the user may choose from a plurality of such devices 10 where each has a body 18 configured at a first end 22 having a diameter yielding an exterior circumference at the first end 22 which is configured to sealably engage with the internal passage 16 of the flexible flange 14. The body 18 may be configured to engage with the internal passage 16 of flexible flanges 14 of the breast pumps 12 of a single manufacturer, where needed, or the pumps 12 sold by multiple different manufacturers. It was found that providing the flange devices 10 having a first sealing ridge 26 and a second sealing ridge 28 both extending in the form of an annular projecting ring from the exterior circumferential surface of the body 18 of the device 10 adjacent the first end 2 thereof, with an annular channel 30 therebetween, allowed for a better seal and fit to the variations in manufacture of provided flanges 14. Such a configuration is thus preferable.

The annular channel 30 between the first sealing ridge 16 and second sealing ridge 28 was found to also better accommodate engagements to provided flanges 14 where suction is communicated off center or from a side. For breast pumps 12 having a side-positioned suction component, a suction opening 32 may be provided which will communicate suction from the pump 12 through this annular channel 30 to the axial passage 20 and to the breast. The suction opening 32 may be provided in different shapes and sizes so long as it is positioned within the channel 30.

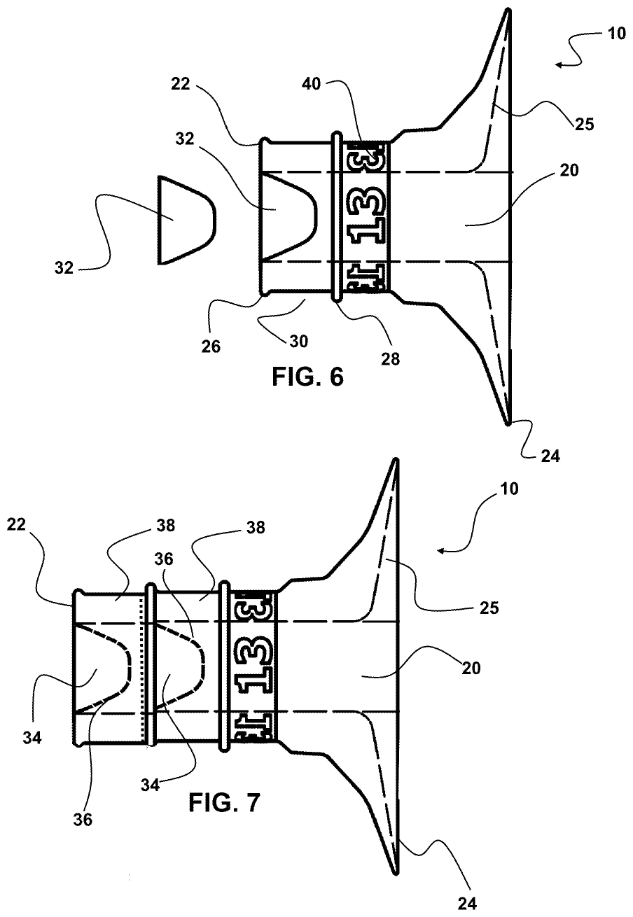

In modes of the device 10 engaged with a pump 12 providing suction to an opening aligned with the axis of the internal passage 16, the suction opening 32 may not be required. However, due to the two sealing ridges 26 and 28, such axial aligned suction will still communicate through the axial passage 20. Consequently, the device 10 can be provided, as shown in FIGS. 6-7, with a removable section 34 which forms a sealed sidewall of the body 18 at the first end 22, when in place. However, along the perimeter edge of the removable section 34 the sidewall may be cut partially with a formed recess 36 (FIG. 7) which will render the removable section 34 frangible such that it may be torn from the sidewall to form the suction opening 32, if needed. If not needed, the removable section 34 would remain in place thereby forming a sealed sidewall, and suction would be communicated from the pump 12 through a passage aligned with the internal passage 16.

Shown in FIG. 7, the flange adapter device 10 herein may also be provided in a configuration with removable end sections 38 positioned at the first end 22. Or a plurality of end sections 38 may be positioned in sequence starting at the first end 22. As shown, the removable sections 34 may also be provided with each such removable end section 38.

The removable end sections 38, if provided, will allow for an adjustment of the length of the body 18 of the device 10 to accommodate the differing length of the internal passages 16 of conventional flexible provided flanges 14 of different pump 12 manufacturers. Where needed, a shortening of the body 18 of the device 10 herein may be accomplished by the removal of one or more removable end sections 38. These removable end sections 38, if provided, will be rendered frangible by formation of a recess or perforations in the same fashion as the removable sections 34 which may be torn from the body 18 to form the suction openings 32 such as a cut forming a recess 36 into the external surface of the body 18.

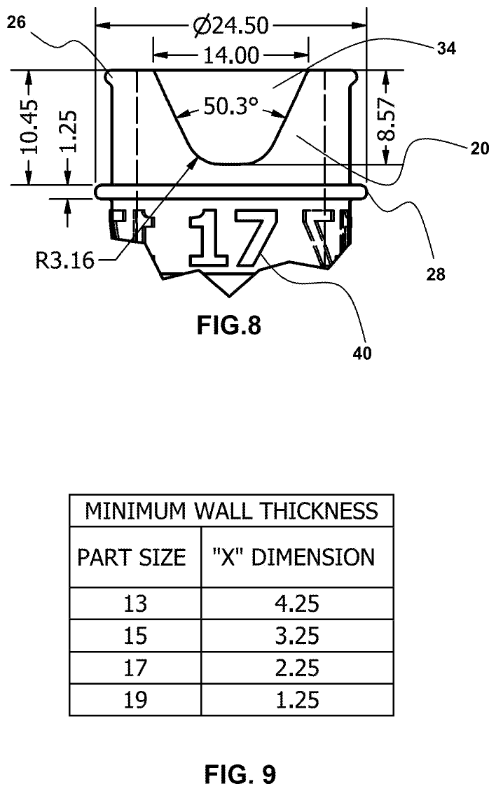

In FIG. 8 is shown an enlargement of the first end of the body 18 forming the flange adapter device 10 herein. Shown are examples of the dimensions of current versions thereof. However, those skilled in the art will realize that such dimensions may change depending on the manufacturer of the pump 12 and the flexible provided flange 14 thereof.

In FIG. 9 is depicted a chart of sidewall thicknesses which may be formed to yield varying diameters D3 of the axial passage 20. However, the external dimensions at the first end 26 of the body 18 may remain static or the same. Maintaining the exterior of the body 18 at the first end 22 thereof thereby configures the first end 22 of each member of a kit having a plurality of flange adapter devices 10 therein, removably sealably engageable with the internal passage 16 of the intended flexible flange 14. Varying the sidewall thickness, however, will vary the diameter D3 of the axial passage 20, thereby allowing for multiple sizes where the user may choose the body 18 which has the diameter D3 of the axial passage 20 which fits their nipple comfortably and allows for communication of suction from the pump 12 to express and store breast milk.

Finally, in a method of employment of the device 10 herein depicted in FIGS. 1-9, the user will first determine a diameter of the nipple of their breast. Such may be determined using rulers or calipers or other available diameter measuring devices. Once a diameter of the nipple is determined, the user will choose the device 10, having the appropriate axial passage 20 diameter D3, that most closely matches their nipple diameter. Indicia 40 identifying the diameter D3 of the axial passage 20 of the body 18 of each individual device 10 available allows the user to choose a matching body 18.

Once the body 18 is chosen from a plurality thereof, having differing axial passage 20 diameters, the first end of the chosen body 18 will be engaged with the internal passage 16 of the flexible flange 14 of the pump 12 manufacturer. Thereafter, the pump 12 would be energized and suction will be communicated to the nipple of a breast in operative engagement at the second end 24 of the chosen body 18 to express milk. Preferably the second end 24 of the body of the adapter device 10 is frusto-conical in shape to provide a large sealing area or sealing flange 25 with the area of the breast of the user surrounding the nipple thereof.

Where the user determines that suction communicated to the internal passage 16 of the flexible flange 14 is from an off center or side-positioned source, if a suction opening 32 is already positioned on the chosen body 18, it will be aligned with the suction conduit of the flexible flange 14. If the suction opening is not present yet, the user will tear the removable section 34 from the sidewall of the body 18, and then position the formed suction opening 32 aligned with the side-positioned suction conduit. Thereafter, the conventional pump 12 is energized.

It should be noted that any of the different depicted and described configurations and components of the breast flange adapter device herein can be employed with any other configuration or component shown and described as part of the device herein. Additionally, while the present invention has been described herein with reference to particular embodiments thereof and/or steps in the method of production or use, a latitude of modifications, various changes and substitutions are intended in the foregoing disclosure, and it will be appreciated that in some instance some features, or configurations, of the invention could be employed without a corresponding use of other features without departing from the scope of the invention as set forth in the following claims. All such changes, alternations and modifications as would occur to those skilled in the art as equivalents are considered to be within the scope of this invention as broadly defined in the appended claims.

Further, the purpose of any abstract of this specification is to enable the U.S. Patent and Trademark Office, the public generally, and especially the scientists, engineers, and practitioners in the art who are not familiar with patent or legal terms or phraseology, to determine quickly from a cursory inspection the nature and essence of the technical disclosure of the application. Any such abstract is neither intended to define the invention of the application, which is measured by the claims, nor is it intended to be limiting, as to the scope of the invention in any way.

What is claimed is:

1. A breast flange adapter apparatus, comprising:
a body, said body being cylindrical and formed of flexible material, said body having a first end opposite a second end and having a sidewall thereof surrounding an axial passage communicating between said first end and said second end of said body;
said first end of said body configured for forming a sealed engagement with an internal passage of a provided flange of a conventional breast pump;
a second end of said axial passage at said second end of said body having a diameter thereof sized for engagement around an exterior circumference of a nipple of a breast of a user; and
said body having a sealing flange at said second end surrounding said axial passage.

2. The breast flange adapter apparatus of claim 2 additionally comprising:
a removable portion of said sidewall positioned at said first end of said body.

3. The breast flange adapter apparatus of claim 1 additionally comprising:
a first annular sealing ridge extending from an exterior surface of said body at said first end of said body;
a second annular sealing ridge extending from said exterior surface of said body; and
an annular channel positioned in between said first annular sealing ridge and said second annular sealing ridge.

4. The breast flange adapter apparatus of claim 2 additionally comprising:
a first annular sealing ridge extending from an exterior surface of said body at said first end of said body;
a second annular sealing ridge extending from said exterior surface of said body;
an annular channel positioned in between said first annular sealing ridge and said second annular sealing ridge; and
said removable portion of said sidewall positioned in said annular channel.

5. The breast flange adapter apparatus of claim 1 having a plurality of said breast flange adapters in a kit thereof;
each respective said breast flange adapters in said kit having a different sized said diameter of said second end of said axial passage; and
wherein a user can choose a respective one of said plurality of breast flange adapters from said kit having a respective sized diameter of said second end of said axial passage which is sized for their respective nipple.

6. The breast flange adapter apparatus of claim 2 having a plurality of said breast flange adapters in a kit thereof;
each respective said breast flange adapters in said kit having a different sized said diameter of said second end of said axial passage; and
wherein a user can choose a respective one of said plurality of breast flange adapters from said kit having a respective sized diameter of said second end of said axial passage which is sized for their respective nipple.

7. The breast flange adapter apparatus of claim 3 having a plurality of said breast flange adapters in a kit thereof;
each respective said breast flange adapters in said kit having a different sized said diameter of said second end of said axial passage; and
wherein a user can choose a respective one of said plurality of breast flange adapters from said kit having a respective sized diameter of said second end of said axial passage which is sized for their respective nipple.

8. The breast flange adapter apparatus of claim 4 having a plurality of said breast flange adapters in a kit thereof;
each respective said breast flange adapters in said kit having a different sized said diameter of said second end of said axial passage; and
wherein a user can choose a respective one of said plurality of breast flange adapters from said kit having a respective sized diameter of said second end of said axial passage which is sized for their respective nipple.

9. The breast flange adapter apparatus of claim 1 additionally comprising:
said body at said first end thereof having a plurality of removable end sections; and
whereby a length of said body between said first end and said second end may be varied by said user by removal of a said end section.

10. The breast flange adapter apparatus of claim 2 additionally comprising:
said body at said first end thereof having a plurality of removable end sections;
each of said removable end sections having a said removable portion of said sidewall positioned; and
whereby a length of said body between said first end and said second end may be varied by said user by removal of a said end section.

11. The breast flange adapter apparatus of claim 3 additionally comprising:
said body at said first end thereof having a plurality of removable end sections;
each of said removable end sections having a said removable portion of said sidewall positioned within an annular channel in-between a first annular sealing ridge extending from an exterior surface of said body and a second annular sealing ridge extending from said exterior surface of said body; and
whereby a length of said body between said first end and said second end may be varied by said user by removal of a said end section.

12. The breast flange adapter apparatus of claim 1 additionally comprising:
indicia positioned on an exterior surface of said body correlating to said diameter of said second end of said axial passage.

13. The breast flange adapter apparatus of claim 5 additionally comprising:
each respective said breast flange adapters in said kit having indicia positioned on an exterior surface of said body thereof, correlating to said diameter of said second end of said axial passage.
